(19) United States
(12) Patent Application Publication
(10) Pub. No.: US 2024/0301091 A1
(43) Pub. Date: Sep. 12, 2024

(54) METHOD OF PREPARATION OF CARBOXYMETHYL CELLULOSE HAVING IMPROVED STORAGE STABILITY

(71) Applicant: NUTRITION & BIOSCIENCES USA 1, LLC, Rochester, NY (US)

(72) Inventors: Sebastian Foertsch, Bomlitz (DE); Rene Kelling, Bomlitz (DE); Oliver Petermann, Bomlitz (DE)

(21) Appl. No.: 18/261,499

(22) PCT Filed: Jan. 28, 2022
(86) PCT No.: PCT/EP2022/052025

§ 371 Date: Jul. 14, 2023

Related U.S. Application Data
(60) Provisional application No. 63/143,145, filed on Jan. 29, 2021.

(51) Int. Cl. C08B 11/12; A23L 29/262; A61K 47/38

(52) U.S. Cl. CPC C08B 11/12; A23L 29/262; A61K 47/38

(57) ABSTRACT

Carboxymethyl cellulose of improved storage stability is produced by a process which comprises the steps of
a) reacting cellulose with an alkalization agent in the presence of water and one or more organic solvents, wherein the total amount of organic solvent(s) is at least 50 weight percent, based on total weight of cellulose, water and organic solvent(s);
b) reacting the alkalized cellulose with monohaloacetic acid or a salt thereof to produce a reaction mixture comprising a carboxymethyl cellulose salt, water and one or more organic solvents;
c) adding acid to the reaction mixture from step b) to produce a carboxymethyl cellulose having a pH of from 6 to 10, when dissolved in water at a concentration of 1 weight percent at a temperature of 25° C., and
d) during and/or after acid addition in step c), subjecting the reaction mixture to shearing at a shear rate of at least 800 s⁻¹.

### FIELD OF THE INVENTION

The present invention is directed to a method of preparing carboxymethyl cellulose (CMC) having increased storage stability, to novel carboxymethyl cellulose and to its uses.

### BACKGROUND OF THE INVENTION

CMCs are used in a wide variety of technical fields. Areas which benefit from the thickening and gel-forming properties of CMC are, for example, oil industry (e.g. in drilling fluids), food industry, pharmaceutical industry, paper industry, electrical and electronic industry, textile industry and construction industry. In the pharmaceutical industry CMC is used in a number of products and for a variety of purposes. Thus, CMC is found in capsules, powders, granules and pellets, as well as tablets and coatings for tablets. It can be used as rheology modifier or for suspension stabilization in semi-solid and liquid dosage forms, including ointments, creams, gels, pastes, solutions, suspensions, emulsions and in wound care products. When used in pharmaceutical products the CMC needs to meet the regulatory requirements for use in pharmaceutical applications, for example as described in the European Pharmacopeia (Ph. Eur. or EP) or in the United States Pharmacopeia (USP). For instance, the European Pharmacopeia requires viscosity stability of CMC in the range of 75% to 140% of the nominal viscosity of the CMC in a 2 wt. % aqueous solution. CMC is also widely used in food products to absorb and hold water, to control crystal growth, as a thickener, as a binder, to increase shelf life, and to provide desired texture or body. Thus, viscosity stability of CMC is also important in food products for quality reasons.

Methods of preparing CMC are well-known to the person skilled in the art and can be found in e. g. U.S. Pat. Nos. 2,067,946; 2,636,879; 4,063,018; 4,250,305; and 4,339,573, International Patent Applications WO 99/20667 and WO 2011/120533 and in Stigsson, Chem. Eng. and Material Proceedings, December 2001, 1-12. In brief, the manufacturing process is as follows. Raw cellulose is fed to a reactor where it is ionized by reaction with NaOH in a mercerization step. The mercerization is followed by an etherification reaction where the alkali cellulose is reacted with monochloroacetic acid (MCA) or sodium monochloroacetate to yield CMC. pH of the reaction mixture may be adjusted by addition of acid. Subsequently the reaction mixture is filtered, the solid residue is washed, dried and ground to provide CMC.

CMCs of a wide range of viscosities are commercially available. It is known that the viscosity of CMC decreases upon long-term storage of the product, particularly of CMC grades that are designated as medium- and high-viscous grades (above 2000 mPa·s and in particular above 10,000 mPa·s viscosity in a 2% solution in water at 25° C.). Thus, there is a risk that the viscosity will go beyond the above-mentioned limits of the regulatory specifications upon long-term storage of CMC, consequently causing disposal of the entire CMC batch. Therefore, viscosity degradation of CMC, in particular for medium- and high-viscous grades is a serious concern, particularly for pharmaceutical and food applications.

CN104761646A claims ‘Preparation method of sodium carboxymethylcellulose with long shelf life.’ CN104761646A does not disclose any stability data of the sodium CMC of the claimed process, and the document does not state how the improved shelf life of sodium carboxymethylcellulose is achieved.

WO2019064633 claims a method for producing carboxymethyl cellulose or a salt thereof which has better storage stability, with less loss of viscosity over long periods of time. The method according to WO2019064633 comprises: an alkali cellulose conversion step (step 1) in which cellulose and an alkali are reacted in the presence of a solvent mixture comprising water and an organic solvent; an etherification step (step 2) in which the alkali cellulose obtained in step 1 is reacted with an etherification agent; and a purification step (step 3) in which the reaction mixture obtained in step 2 is washed and dried, wherein said method is characterized in that, in step 1, the alkali is used in an amount of 2.0 mol to 2.5 mol and the water is used in an amount of 10 mol to 15 mol per mol anhydroglucose units constituting the cellulose, and the concentration of the alkali relative to the water is 0.5 mol % to 1 mol %. In step 1 the pH is 9 or more, preferably 11 or more, more preferably 12 or more. The upper limit of pH is 14 or less, preferably 13.5 or less. The purification step (step 3) may include a solvent removal step, a washing step, and a drying step. In addition, a pH adjustment step may be further included prior to the solvent removal step. According to Examples 1-9 the pH in step 1 is 12.90-13.89 and 1500-2750 g of a solvent (isopropyl alcohol/water=80/20 (mass ratio)), are used per 550 g of cellulose in chip form. When the amount of alkali added to the cellulose is large (comparative example 1, pH=14.55), when it is small (comparative example 2, pH=12.20), when the amount of water added is large (comparative example 3), or when it is small (comparative example 4), the decrease in viscosity becomes large. In all Examples and Comparative Examples, the pH in step 3 is adjusted to about 9.5 using aqueous acetic acid and the reaction solvent is evaporated to obtain crude sodium CMC. The crude sodium CMC is adjusted to pH 9.5 by adding sodium hydroxide and subsequently washed using a methanol/water mixture. Unfortunately, the taught pH range of 9 to 14, such as 9.5, in the pH adjustment step 3 does not comply with various regulatory requirements, such as the United States Pharmacopeia requiring a pH 6.5-8.5 in 1% aqueous solutions at 25° C.

JP2008019344 discloses a method to provide a partial acid-type CMC having little reduction in viscosity, comprising the steps of (a) forming an alkali cellulose from a raw material pulp and then producing a carboxymethyl cellulose salt by etherification of the alkali cellulose, (b) converting the carboxymethyl cellulose salt into an acid-type carboxymethyl cellulose, (c) cleaning the acid type carboxymethyl cellulose, and (d) reacting the cleaned acid-type carboxymethyl cellulose with an alkali, such as alkali hydroxide. The amount of alkali added is 5 to 10 times the weight necessary for achieving the theoretical acid etherification degree.

There is a need in the art for further methods for preparation of CMC having improved storage stability. Thus, the object to be solved by the present invention is to provide a new process for preparing CMC having improved storage stability, particularly a versatile process that is not limited to the production of a CMC salt having a high pH when dissolved in water.

### SUMMARY OF THE INVENTION

Surprisingly, it has been found that the loss of viscosity of CMC upon storage is diminished if acid is added to the reaction mixture after the etherification step, which comprises alkali CMC, to produce a CMC having a pH of from 6 to 10, when dissolved in water at a concentration of 1 weight percent at a temperature of 25° C., and the reaction mixture during and/or after the acid addition is subjected to a shear rate of at least 800 s⁻¹.

Accordingly, one aspect of the present invention is a process of preparing carboxymethyl cellulose comprising the steps of
a) reacting cellulose with an alkalization agent in the presence of water and one or more organic solvents, wherein the total amount of organic solvent(s) is at least 50 weight percent, based on total weight of cellulose, water and organic solvent(s);
b) reacting the alkalized cellulose with monohaloacetic acid or a salt thereof to produce a reaction mixture comprising a carboxymethyl cellulose salt, water and one or more organic solvents;
c) adding acid to the reaction mixture from step b) to produce a carboxymethyl cellulose having a pH of from 6 to 10, when dissolved in water at a concentration of 1 weight percent at a temperature of 25° C., and
d) during and/or after acid addition in step c), subjecting the reaction mixture to shearing at a shear rate of at least 800 s⁻¹.

Another aspect of the present invention is a carboxymethyl cellulose obtainable by the above-mentioned process.

Yet another aspect of the present invention is a carboxymethyl cellulose having a pH of 6 to 10, when dissolved in water at a concentration of 1 weight percent at a temperature of 25° C., and exhibiting a decrease in viscosity, measured as a 1 weight percent solution in water at 25° C.,
of less than 30 percent after 8 weeks of storage at 75% relative humidity and a temperature of 40° C., and
of less than 40 percent after 12 weeks of storage at 75% relative humidity and a temperature of 40° C.

Yet another aspect of the present invention is a use of the above-mentioned carboxymethyl cellulose as a thickening agent, gelling agent, binder, stabilizer, emulsifier, film forming agent, suspending agent, protective colloid, or crystallization inhibitor.

Yet another aspect of the present invention is a use of the above-mentioned carboxymethyl cellulose in pharmaceutical dosage forms or in food products.

### DETAILED DESCRIPTION OF THE INVENTION

The term “CMC” or “carboxymethyl cellulose” as used herein encompasses cellulose substituted with groups of the formula —CH₂CO₂A, wherein A is hydrogen or a monovalent cation, such as K⁺ or preferably Na⁺. The term “CMC” or “carboxymethyl cellulose” therefore encompasses carboxymethyl cellulose comprising free acid groups as well as ‘sodium carboxymethyl cellulose’ and ‘sodium CMC’, potassium carboxymethyl cellulose’ and ‘potassium CMC’.

The term “carboxymethyl cellulose salt” as used herein encompasses cellulose substituted with groups of the formula —CH₂CO₂A′, wherein A′ is a monovalent cation, such as K⁺ or preferably Na⁺. The term “sodium carboxymethyl cellulose” or “sodium CMC” as used herein encompasses cellulose substituted with groups of the formula —CH₂COO⁻Na⁺.

The CMCs prepared according to the method of the present invention generally has a degree of substitution (DS) of from 0.1 to 1.6, preferably from 0.15 to 1.5, such as from 0.6 to 1.2, or even such as from 0.7 to 1.1. The degree of substitution is the average number of OH groups that have been substituted in one anhydroglucose unit, thus the theoretically maximum DS value is 3. It can be determined according to ASTM D 1439-03 (Reapproved 2008) “Standard Test Methods for Sodium Carboxymethylcellulose; Degree of Etherification, Test Method B: Nonaqueous Titration”. The treatment of a solid sample of the CMC with glacial acetic acid at boiling temperature releases an acetate ion quantity equivalent to the sodium carboxymethyl groups. These acetate ions can be titrated as a strong base in anhydrous acetic acid using a perchloric acid standard solution. The titration end point is determined potentiometrically. Other alkaline salts of carboxylic acids (e. g. sodium glycolate and di-sodium diglycolate) behave similarly and are co-titrated.

The CMCs prepared according to the method of the present invention have a viscosity, measured as a 1 weight percent (wt.-%) solution in water, of preferably at least 100 mPa·s, more preferably at least 250 mPa·s, even more preferably at least 500 mPa·s, and most preferably at least 1000 mPa·s. The viscosity is preferably up to 20,000 mPa·s, more preferably up to 15,000 mPa·s, even more preferably up to 10,000 mPa·s, and most preferably up to 6,000 mPa·s, measured as a 1 wt.-% solution in water at 25° C.

In order to determine the viscosity, a 1 wt.-% solution in water is prepared as follows (total amount of solution 350 g). 346.5 g deionized water (water in CMC is subtracted) is placed in a 500 ml screw cap bottle. The bottle is placed in a tempering bath at a temperature of 25° C. 3.5 g (dry weight) of the CMC are added onto the surface. The product sample is stirred at a constant rotating speed (approx. 1000-1500 rpm) and at a temperature of 25° C. for 1 h and 30 min. Then, the stirrer is switched off and the solution is kept at 25° C. without stirring for 30 minutes before the viscosity is determined. The viscosity is analyzed using a Brookfield viscometer (LVT) at 25° C. (+/−0.1° C.) as described in ASTM D 1439-03 (Reapproved 2008). As listed in Table 1 of ASTM D 1439-03, viscosities in the range of 100 to 200 mPa·s are measured at 30 rpm using spindle No. 1, viscosities in the range of 200 to 1000 mPa·s are measured at 30 rpm and spindle No. 2; viscosities in the range of 1000 to 4000 mPa·s are measured at 30 rpm and spindle No. 3; and viscosities in the range of 4000 to 10000 mPa·s are measured at 30 rpm and spindle No. 4.

The type of cellulose to be used for preparing the CMC is not essential for the present invention and is governed by the intended end-use of the CMC. Conventional starting materials are natural cellulose, such as cotton linters and wood pulp, e.g. hard wood or soft wood pulp. Typically, cotton linters or wood pulp are used, depending on the desired application of the CMC.

Methods of preparing CMC are well-known to the person skilled in the art and can be found in e. g. U.S. Pat. Nos. 2,067,946; 2,524,024; 2,636,879; 4,063,018; 4,250,305; and 4,339,573, in International Patent Application published as WO 2011/120533 and in Stigsson, Chem. Eng. and Material Proceedings, December 2001, 1-12.

The essential steps of the preparation method are alkalizing the cellulose by reaction with the alkalizing agent (alkalizing step (a)) and adding monohaloacetic acid to cause etherification of the alkali cellulose (carboxymethylation step (b)), adding acid to the reaction mixture from step b) to produce a carboxymethyl cellulose having a pH of from 6 to 10, when dissolved in water at a concentration of 1 wt.-% at a temperature of 25° C. (acid addition step c)) and d) during and/or after acid addition in step c), subjecting the reaction mixture to a shear rate of at least 800 s⁻¹.

The cellulose is first alkalized in the presence of water and one or more organic solvents in the alkalizing step a) and then the monohaloacetic acid is added in the carboxymethylation step b). Preferred organic solvents are alcohols having 1 to 4 carbon atoms, such as methanol, ethanol, n-propyl alcohol, isopropyl alcohol, n-butyl alcohol or isobutyl alcohol, ketones having 3 to 5 carbon atoms, such as acetone or methyl ethyl ketone, or mixtures of two or more of these solvents, or mixtures of one or more of the foregoing solvents with aromatic hydrocarbons having 6 to 8 carbon atoms, such as benzene, toluene or xylene. Preferably isopropyl alcohol or a combination of isopropyl alcohol and methanol is used as organic solvent(s). When using a combination of isopropyl alcohol and methanol as the organic solvents, the amount of isopropanol is generally from 50 to 99 wt.-%, preferably from 80 to 98 wt.-%, more preferably from 70 to 98 wt.-%, and most preferably from 85 to 97 wt.-%, based on the total weight of isopropanol and methanol.

The amount of cellulose in step a) is preferably within the range of from 1 to 25 wt.-%, more preferably from 2 to 12 wt.-%, even more preferably from 3 to 10 wt.-%, and most preferably from 4 to 8 wt.-%, each based on the total weight of cellulose, water and organic solvent(s).

The amount of water in step a) is preferably within the range of from 1 to 25 wt.-%, more preferably from 2 to 20 wt.-%, even more preferably from 3 to 15 wt.-%, and most preferably from 4 to 12 wt.-%, each based on the total weight of cellulose, water and organic solvent(s).

The total amount of one or more organic solvents in step a) is at least 50 wt.-%, based on total weight of cellulose, water and organic solvent(s). Preferably, the total amount of one or more organic solvents in step a) is within the range of from 50 to 98 wt.-%, more preferably 68 to 96 wt.-%, even more preferably from 75 to 94 wt.-%, and most preferably from 80 to 92 wt.-%, each based on the total weight of cellulose, water and organic solvent(s).

The alkalizing agent is preferably an alkali metal hydroxide, such as NaOH, KOH or a mixture thereof. In one embodiment of the invention NaOH is used as the alkalization agent. In another embodiment KOH is used as the alkalization agent. In the latter case remaining K⁺ ions in the produced CMC are preferably exchanged by Na⁺ ions in a subsequent step. The amount of the alkalizing agent depends on the desired degree of substitution. Preferably the molar ratio between alkalizing agent and anhydroglucose units of the cellulose is from 0.5:1 to 5:1, more preferably from 0.7:1 to 3.5:1, and most preferably from 1.0:1 to 2.6:1.

In the alkalizing step the single components (e.g. cellulose, water, organic solvent(s), alkalizing agent) can be added in any order. However, it is preferred that the cellulose is combined with water and one or more organic solvents in a first step and the alkalizing agent is then added in a subsequent step. If a combination of isopropyl alcohol and methanol is used as organic solvent, methanol can be added to the reaction mixture before, at the same time or shortly after the addition of the monohaloacetic acid, but preferably it is added at the same time as water and/or isopropyl alcohol, either separately or as a mixture with water and/or isopropyl alcohol.

There are various options how to contact the cellulose with the alkalizing agent. Preferably, cellulose powder is used as starting material. Typically, the cellulose powder is suspended (slurried) in a solvent/water mixture comprising water, and one or more organic solvents as described above before the alkalizing agent is added as a solid or as an aqueous solution. An aqueous solution is preferred that comprises the alkalizing agent, such as an alkali metal hydroxide, preferably NaOH, at a concentration of from 35 to 50 wt.-%, based on the total weight of the aqueous solution.

According to the slurry method the alkalizing step (a) is typically conducted at a temperature within the range of from 10 to 80° C., preferably from 15 to 40° C., more preferably from 15 to 30° C., even more preferably from 18 to 25° C., and most preferably at about 20° C. Typical reaction times for the alkalization step range from 15 min to 4 hours, preferably from 40 to 120 min, and more preferably from 50 to 80 min depending on the reaction temperature. In one embodiment the alkalizing step is conducted at about 20° C. for 50 to 70 min, preferably for 60 min.

In the carboxymethylation step (b) the monohaloacetic acid or a salt thereof, preferably the sodium salt, can be added neat or as a solution, preferably as an aqueous solution or as a solution in water and isopropyl alcohol and optionally methanol. The monohaloacetic acid can be used as free acid or in its salt form. Preferably, monochloroacetic or its salt is used in the inventive process, including all preferred embodiments. The monohaloacetic acid added in step b) of the process is generally from 0.4 to 2.5 mols, preferably from 0.5 to 1.8 mols and more preferably from 0.6 to 1.5 mols per mol of anhydroglucose units of the cellulose. The monohaloacetic acid or a salt thereof added in step b) is generally from 0.25 to 0.7 mols, preferably from 0.4 to 0.6 mols and more preferably from 0.45 to 0.55 mols per mol of mol of alkalizing agent, such as an alkali metal hydroxide, added in step a).

The carboxymethylation step is typically conducted at a temperature within the range of from 40 to 100° C., preferably from 50 to 90° C., more preferably from 60 to 80° C., and most preferably at about 70° C. In preferred embodiments the monohaloacetic acid is already added to the reaction mixture before the carboxymethylation temperature is reached; more preferably the monohaloacetic acid is already added before the heating-up phase begins or within the first minutes of the heating-up phase, generally at a temperature which is described above for the alkalizing step, e.g. at from 15 to 40° C. The early addition of the monohaloacetic acid at lower temperatures may avoid an undesired degradation of the cellulose (resulting in a decrease of viscosity) which may occur at the higher temperatures in absence of monohaloacetic acid. After the desired temperature of the carboxymethylation step (carboxymethylation temperature) as mentioned before is reached, the carboxymethylation temperature is typically held for a period of from 0 to 180 min, preferably from 20 to 140 min.

In steps a) and b) the amounts of cellulose, organic solvent(s), water alkalizing agent, and monohaloacetic acid are preferably chosen as described above. Preferably, the total amount of the organic solvent(s) in the reaction mixture of step b) is at least 50 wt.-%, more preferably at least 60 wt.-%, and most preferably at least 70 wt.-%, based on total weight of the reaction mixture. Preferably, the total amount of the organic solvent(s) in the reaction mixture of step b) is up to 95 wt.-%, more preferably up to 90 wt.-%, and most preferably up to 85 wt.-%, based on total weight of the reaction mixture.

The product obtained in the carboxymethylation step b) is a carboxymethyl cellulose salt, typically the sodium or potassium salt of carboxymethyl cellulose, depending on which alkalizing agent was employed. In the reaction mixture obtained in step b) the amount of the carboxymethyl cellulose salt is generally up to 30 wt.-%, based on total weight of the reaction mixture. The amount of carboxymethyl cellulose salt in the reaction mixture of step b) is preferably within the range of from 1.5 to 25 wt.-%, more preferably from 2.5 to 15 wt.-%, even more preferably from 4 to 12 wt.-%, and most preferably from 5 to 10 wt.-%, based on total weight of the reaction mixture.

According to step c) of the process of the present invention, acid is added to the reaction mixture from step b) to produce a carboxymethyl cellulose (CMC) having a pH of from of 6 to 10, preferably from 6 to 9, more preferably from 6.5 to 8.5, and most preferably from 7 to 8, such as from 7.2 to 7.8, when the CMC is dissolved in water at a concentration of 1 wt.-% at a temperature of 25° C. Preferably an organic acid is added, such as acetic acid or formic acid, more preferably acetic acid. For pH determination in step c) of the process, the CMC is removed from the reaction mixture and washed with an organic solvent or a mixture of one or more organic solvents with water. Preferred organic solvents are those listed above in the description of the alkalizing step a). The most preferred organic solvents, mixtures of organic solvents, and mixtures of organic solvent(s) with water are methanol, acetone, methanol/water mixtures, acetone/methanol mixtures, and methanol/isopropyl alcohol/water mixtures. The CMC is then dissolved at a concentration of 1 wt.-% in water and the pH of the resulting solution of the CMC is measured at 25° C.

During and/or after acid addition in step c), the reaction mixture is subjected to shearing with a shear rate of at least 800 s⁻¹, preferably at least 1500 s⁻¹, more preferably at least 3000 s⁻¹, and most preferably at least 8000 s⁻¹. The shear rate is generally up to 200,000 s⁻¹, and typically up to 100,000 s⁻¹, more typically up to 60,000 s⁻¹ and most typically up to 40,000 s⁻¹. The mentioned shear rate can be obtained in a high shear device, such as a high shear mixer, also known as rotor-stator mixer or homogenizer, or high shear pump. A high shear device commonly comprises a rotor in combination with a stationary part of the shear device, also referred to as “stationary”, such as a stator or housing. The stationary creates a close-clearance gap between the rotor and itself and forms a high-shear zone for materials in this gap. The stationary can include single or multiple rows of openings, gaps or teeth to induce a kind of shear frequency and increased turbulent energy.

One metric for the degree or thoroughness of mixing is the shearing force generated by a mixing device with a high tip speed. Fluid undergoes shear when one area of fluid travels with a different velocity relative to an adjacent area. The tip speed of the rotor is a measure of the kinetic energy generated by the rotation according to the formula:

Tip speed=rotation rate of rotor×rotor circumference.

The shear rate is based on the inverse relationship between the gap distance between the rotor and the stationary part of the shear device which is commonly referred to as the stator or housing. In the case the high shear device is not equipped with a stator, the inner wall of a precipitation vessel serves as a stator.

Shear rate=Tip speed/gap distance between outer diameter of rotor and stationary.

The above-described shearing in step c) is preferably conducted during a time period of from 15 seconds to 15 minutes, more preferably from 30 seconds to 5 minutes, most preferably from 60 seconds to 3 minutes.

In step d) of the process of the present invention, shearing is preferably conducted in a shear device running at a tip speed of at least 4 m/s, preferably at least 8 m/s, and more preferably at least 12 m/s. The tip speed is generally up to 50 m/s, typically up to 30 m/s, and more typically up to 20 m/s.

A further shearing is induced by a velocity difference between the tip velocity of the fluid at the outside diameter of the rotor and the velocity at the centre of the rotor.

High shear devices are also called high shear mixers and encompass different geometries such as axial-discharge and radial-discharge rotor stator mixers (Atiemo-Obeng, V. A. and Calabrese, R. V., 2004. “Rotor-stator mixing devices” in Handbook of Industrial Mixing: Science and Practice, E. L. Paul, V. A. Atiemo-Obeng and S. M. Kresta, John Wiley & Sons, Hoboken, New Jersey, USA.). High shear mixers include ring layer mixers, Ploughshare mixers, Schugi mixers, and Turbulizer mixers. In a preferred embodiment, the high shear mixer is a ring layer mixer. A ring layer mixer generally comprises a horizontal drum with a mixing shaft axially disposed in it. The mixing shaft has blades, bolts, and/or paddles protruding from it. Mixing shaft geometry can create various mixing zones for transporting, dispersing, mixing, and the like. The reaction mixture to be mixed forms a concentric ring via centrifugal force and moves through the mixer in plug-like flow. A suitable ring layer mixer can be procured from Loedige (Paderborn, Germany), under the tradename CORIMIX CM 20.

The CMC is then separated from the reaction mixture and purified, if necessary, depending on the intended end-use, and dried. Purification is performed according to standard methods well-known to the person skilled in the art. For example, the CMC can be washed with organic solvents including mixtures of organic solvents and solvent(s)/water mixtures, such as methanol, acetone, methanol/water mixtures, acetone/methanol mixtures, and methanol/isopropyl alcohol/water mixtures.

As indicated above, shearing at a high shear rate can be conducted during and/or after acid addition in step c). Accordingly, shearing at a high rate as described above can be conducted during acid addition in step c), during the subsequent washing of the CMC or during both steps. Most preferably, shearing at a high rate as described above is conducted during acid addition in step c) or during both the steps of acid addition in step c) and subsequent washing of the CMC.

The improved property of the CMCs prepared according to the method of the present invention is their increased storage stability. The CMCs exhibit a surprisingly low decrease in viscosity over time even at low pH, as stated below.

Hence, another aspect of the present invention are novel carboxymethyl celluloses which have a pH of 6 to 10, when dissolved at a concentration of 1 wt.-% in water at a temperature of 25° C., and which exhibit a decrease in viscosity, measured as a 1 wt.-% solution in water at 25° C., of less than 30 percent after 8 weeks of storage and less than 40 percent after 12 weeks of storage. In some embodiments of the invention, the decrease in viscosity of the CMC, measured as a 1 wt.-% solution in water at 25° C., is even less than 20 percent after 8 weeks of storage, and/or less than 35 percent after 12 weeks of storage. Moreover, in some embodiments of the invention, the decrease in viscosity of the CMC, measured as a 1 wt.-% solution in water at 25° C., is less than 10 percent after 4 weeks of storage. The term “storage” as used herein means storage at 75% relative humidity (RH) and a temperature of 40° C. Storage is conducted at atmospheric pressure in air.

A 1 wt.-% solution of the novel carboxymethyl cellulose in water at a temperature of 25° C. has a pH of 6 to 10, preferably from 6 to 9, more preferably from 6.5 to 8.5, and most preferably from 7 to 8, such as from 7.2 to 7.8.

The pH of the 1 wt.-% solution of the carboxymethyl cellulose in water can be measured using a standard single-rod pH meter.

The novel carboxymethyl cellulose preferably has an initial viscosity, measured as a 1 wt.-% solution in water at 25° C., of at least 100 mPa·s, more preferably at least 250 mPa·s, even more preferably at least 500 mPa·s, and most preferably at least 1000 mPa·s. The viscosity is preferably up to 20,000 mPa·s, more preferably up to 15,000 mPa·s, even more preferably up to 10,000 mPa·s, and most preferably up to 6,000 mPa·s, measured as a 1 wt.-% solution in water at 25° C. The viscosity is analyzed using a Brookfield viscometer as described in ASTM D 1439-03 (Reapproved 2008). More details of the viscosity measurement are described further above. By “initial” viscosity is meant the viscosity measured within one day after its production.

The novel carboxymethyl cellulose generally has a degree of substitution (DS) of from about 0.1 to 1.6, preferably from 0.15 to 1.5, such as from 0.6 to 1.2, or even such as from 0.7 to 1.1. The DS can be determined as described further above.

The CMCs according to the present invention can be used for a many different applications. For example, they can be used as thickening agents and/or gelling agents, binders, stabilizers, emulsifiers, film forming agents, suspending agents, protective colloids, or crystallization inhibitors. Their beneficial properties make the present CMCs particularly useful in pharmaceutical dosage forms, such as capsules, tablets, solutions, suspensions, emulsions, creams, and lotions. The present CMCs are also useful in food products including pet food. For example, the CMCs are useful in the preparation of fruit-based products, of frozen desserts like ice cream or sherbets, in protein-containing products, such as milk products, or in processed meat products, such as sausages.

The following examples are provided for illustrative purposes only and are not intended to limit the scope of the claims that follow. Unless otherwise indicated, all parts and percentages are by weight.

### EXAMPLES

pH Measurement: In step c) of the process, acid is added to the reaction mixture from step b) to produce a carboxymethyl cellulose having a pH of from 6 to 10, when dissolved in water at a concentration of 1 wt.-% at a temperature of 25° C. In Example 1 and Comparative Example A the utilized acid is acetic acid. After acid addition solid carboxymethyl cellulose is recovered from the reaction mixture, washed with methanol and dissolved in water at a concentration of 1 wt.-%. The pH of the resulting solution is measured at a temperature of 25° C.

Viscosity: To determine the viscosity of the carboxymethyl cellulose, a 1 wt.-solution in water can be prepared as described above in the general description. The 1 wt.-solution of the carboxymethyl cellulose in water is analyzed using a Brookfield viscometer (LVT) at 25° C. (+/−0.1° C.). The rotating speed of 30 rpm is chosen. For viscosities in the range of 1000 to 4000 mPa·s spindle No. 3 is chosen; for viscosities in the range of 4000 to 10000 mPa·s spindle No. 4 is chosen.

Viscosity decrease: To determine the decrease in viscosity of the CMC over time, 10 g of the produced CMC powder is put in a 100 ml container. The container is stored in a climate chamber in air at atmospheric pressure at 75% relative humidity (RH) and a temperature of 40° C. such that each portion of CMC is equally exposed to temperature and RH.

The degree of substitution (DS) of the carboxymethyl cellulose can be determined as described above in the general description.

### Example 1

The applied cellulose pulp possessed a dry content of 94.03% and an intrinsic viscosity of 1600 mL/g. Prior to application it was ground with a cutting mill using 400 micron Conidur screens.

Ground cellulose (0.8 mol, atro) was suspended in a dry lab reactor (3 L) with 1.96 kg of a mixture of isopropanol (88.3 wt.-%), methanol (4.7 wt.-%) and water (7.0 wt.-%). The amount of cellulose in the slurry (sum of pulp, isopropanol, methanol and water) was 6.5 wt.-%. For the overall water content in the slurry the amount of water in the applied pulp and caustic was also considered. The vessel was inertisized three times with nitrogen (each time applied vacuum of approximately 250 mbar) and purged with a constant low nitrogen gas flow during the whole synthesis. The stirrer was started and NaOH (2.2 mol/mol anhydroglucose unit (AGU)) was added. Subsequently, alkalization at 20° C. for 60 min was performed. Chloroacetic acid (1.1 mol/mol AGU) was added and the mixture was heated to 70° C. in 40 min. Etherification was performed at 70° C. for 120 min, and the mixture was cooled to 20° C. in approximately 40 min.

The pH of the carboxymethyl cellulose, determined as a 1 wt.-% solution in water at a concentration of at 25° C., was adjusted to approximately 7.5 by addition of acetic acid to the reaction mixture. Upon addition of acetic acid, the reaction mixture was subjected to a high shear treatment at a shear rate of 18840 s⁻¹ for 2 minutes using an Ultra-Turrax high shear mixer.

The suspension was filtered. The solid residue was washed with a mixture of methanol/isopropanol/water (50/30/20 vol %) until no chloride could be detected anymore in the filtrate. The latter test was performed by addition of AgNO₃ to a sample, whereas chloride forms insoluble white AgCl. Commonly, the raw product had to be washed five times with 2 L of the mixture to become free of chloride. Finally, the product was washed with pure methanol (2 L), dried in an air-swept dry cabinet at 55° C. over night and ground in a laboratory mixer.

### Comparative Example A

Comparative Example A was prepared as described for Example 1 above, except that the high shear treatment described in Example 1 was not carried out.

The CMCs of Example 1 and Comparative Example A were analyzed as described above. Their properties are listed in Table 1 below. Their viscosities over extended time periods were measured and the viscosity reductions over these time periods were calculated. The results are listed in Table 2 below.

TABLE 1

| Sample | Comparative Example A | Example 1 |
|---|---|---|
| Dry content [%] | 95.24 | 93.99 |
| Brookfield viscosity [mPas] | 4780 | 4840 |
| DS | 0.89 | 0.93 |
| NaCl [%] | <0.05 | <0.05 |
| pH | 7.4 | 7.7 |

TABLE 2

| | | Within 1 | | | |
|---|---|---|---|---|---|
| | | day after | After 4 | After 8 | After 12 |
| | | synthesis | weeks | weeks | weeks |
| Comparative | Dry content [%] | 95.24 | 72.79 | 74.43 | 73.55 |
| Example A | Brookfield viscosity | 4780 | 4200 | 3200 | 2820 |
| | [mPas] | | | | |
| | Relative viscosity [%] | 100% | 88% | 67% | 59% |
| | Viscosity reduction [%] | 0% | 12% | 33% | 41% |
| Example 1 | Dry content [%] | 93.99 | 74.59 | 74.60 | 73.32 |
| | Brookfield viscosity | 4840 | 4500 | 4060 | 3260 |
| | [mPas] | | | | |
| | Relative viscosity [%] | 100% | 93% | 84% | 67% |
| | Viscosity reduction [%] | 0% | 7% | 16% | 33% |

1. A process of preparing carboxymethyl cellulose comprising the steps of
a) reacting cellulose with an alkalization agent in the presence of water and one or more organic solvents, wherein the total amount of organic solvent(s) is at least 50 weight percent, based on total weight of cellulose, water and organic solvent(s);
b) reacting the alkalized cellulose with monohaloacetic acid or a salt thereof to produce a reaction mixture comprising a carboxymethyl cellulose salt, water and one or more organic solvents;
c) adding acid to the reaction mixture from step b) to produce a carboxymethyl cellulose having a pH of from 6 to 10, when dissolved in water at a concentration of 1 weight percent at a temperature of 25° C., and
d) during and/or after acid addition in step c), subjecting the reaction mixture to shearing at a shear rate of at least 800 s⁻¹.

2. The process of claim 1 wherein in step a) the molar ratio between alkalizing agent and anhydroglucose units of the cellulose is from 0.5:1 to 5:1.

3. The process of claim 1 wherein in step a) the amount of cellulose is from 1 to 25 weight percent, based on the total weight of cellulose, water and one or more organic solvent(s).

4. The process of claim 1 wherein in step a) the amount of water is from 1 to 25 weight percent, based on the total weight of cellulose, water and one or more organic solvent(s).

5. The process of claim 1 wherein in step a) the organic solvent is an alcohol having 1 to 4 carbon atoms, a ketone having 3 to 5 carbon atoms, or a mixture of two or more of said solvents, or a mixture of one or more of said solvents with an aromatic hydrocarbon having 6 to 8 carbon atoms.

6. The process of claim 1 wherein step a) is conducted in the presence of water, isopropyl alcohol and methanol.

7. The process of claim 1 wherein in the reaction mixture obtained in step b) the amount of carboxymethyl cellulose salt is up to 30 weight percent, based on the total weight of the reaction mixture in step b).

8. The process of claim 1 wherein in step d) the reaction mixture is subjected to shearing at a shear rate of at least 1500 s.

9. The process of claim 1 wherein the produced carboxymethyl cellulose has a viscosity of from 100 to 20,000 mPa·s, measured as a 1 weight percent solution in water at 25° C.

10. The process of claim 1 wherein the produced carboxymethyl cellulose has a viscosity of from 500 to 10,000 mPa·s, measured as a 1 weight percent solution in water at 25° C.

11. Carboxymethyl cellulose obtainable by the process of claim 1.

12. Carboxymethyl cellulose having a pH of 6 to 10, when dissolved in water at a concentration of 1 weight percent at a temperature of 25° C., and exhibiting a decrease in viscosity, measured as a 1 weight percent solution in water at 25° C.,
of less than 30 percent after 8 weeks of storage at 75% relative humidity and a temperature of 40° C., and
of less than 40 percent after 12 weeks of storage at 75% relative humidity and a temperature of 40° C.

13. The carboxymethyl cellulose of claim 12 having an initial a viscosity of from 100 to 20,000 mPa·s, measured as a 1 weight % solution in water at 25° C.

14. A thickening agent, gelling agent, binder, stabilizer, emulsifier, film forming agent, suspending agent, protective colloid, or crystallization inhibitor, wherein the thickening agent, gelling agent, binder, stabilizer, emulsifier, film forming agent, suspending agent, protective colloid, or crystallization inhibitor comprises the carboxymethyl cellulose of claim 11.

15. A pharmaceutical dosage form or food product, wherein the pharmaceutical dosage form or food product comprises the carboxymethyl cellulose of claim 11.

16. A thickening agent, gelling agent, binder, stabilizer, emulsifier, film forming agent, suspending agent, protective colloid, or crystallization inhibitor, wherein the thickening agent, gelling agent, binder, stabilizer, emulsifier, film forming agent, suspending agent, protective colloid, or crystallization inhibitor comprises the carboxymethyl cellulose of claim 12.

17. A pharmaceutical dosage form or food product, wherein the pharmaceutical dosage form or food product comprises the carboxymethyl cellulose of claim 12.
